(19) United States
(12) Patent Application Publication
(10) Pub. No.: US 2026/0153443 A1
(43) Pub. Date: Jun. 4, 2026

(54) DEPTH-COMPENSATED IMAGE ANALYSIS USING MULTIPLE LIGHT SIGNATURES

(71) Applicant: Precision Healing LLC, Newton, MA (US)

(72) Inventors: David B Strasfeld, Somerville, MA (US); Michael Anthony DeLuca, Lowell, MA (US); Mari Saif, Gloucester, MA (US)

(73) Assignee: Precision Healing LLC, Newton, MA (US)

(21) Appl. No.: 19/457,317

(22) Filed: Jan. 23, 2026

Related U.S. Application Data
(63) Continuation-in-part of application No. 19/045,608, filed on Feb. 5, 2025.
Application No. 19/045,608 is a continuation-in-part of application No. 18/369,870, filed on Sep. 19, 2023.
Application No. 18/369,870 is a continuation-in-part of application No. 17/564,318, filed on Dec. 29, 2021.
Application No. 17/564,318 is a continuation-in-part of application No. 17/155,141, filed on Jan. 22, 2021, now Pat. No. 12,053,262.
(60) Provisional application No. 62/964,969, filed on Jan. 23, 2020.
(60) Provisional application No. 63/132,541, filed on Dec. 31, 2020.
(60) Provisional application No. 63/408,336, filed on Sep. 20, 2022.
(60) Provisional application No. 63/451,247, filed on Mar. 10, 2023.
(60) Provisional application No. 63/550,096, filed on Feb. 6, 2024.
(60) Provisional application No. 63/750,808, filed on Jan. 29, 2025.

(51) Int. Cl. G01N 21/64; G01B 11/24; G01N 21/55; G01S 17/08; G06T 7/00; G06T 7/521; G06T 7/80

(52) U.S. Cl. CPC G01N 21/6456; G01B 11/24; G01N 21/55; G01N 21/6486; G01S 17/08; G06T 7/0012; G06T 7/521; G06T 7/80; G06T 2200/04; G06T 2207/10024; G06T 2207/10064; G06T 2207/20081; G06T 2207/30004

(57) ABSTRACT

Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

### RELATED APPLICATIONS

This application claims the benefit of U.S. provisional patent application “Depth-Compensated Image Analysis Using Multiple Light Signatures” Ser. No. 63/750,808, filed Jan. 29, 2025.

This application is also a continuation-in-part of U.S. patent application “Wound Care Image Analysis Using A Smartphone” Ser. No. 19/045,608, filed Feb. 5, 2025, which claims the benefit of U.S. provisional patent applications “Wound Care Image Analysis Using A Smartphone” Ser. No. 63/550,096, filed Feb. 6, 2024, and “Depth-Compensated Image Analysis Using Multiple Light Signatures” Ser. No. 63/750,808, filed Jan. 29, 2025.

The U.S. patent application “Wound Care Image Analysis Using A Smartphone” Ser. No. 19/045,608, filed Feb. 5, 2025, is also a continuation-in-part of U.S. patent application “Wound Care Image Analysis Using Light Signatures” Ser. No. 18/369,870, filed Sep. 19, 2023, which claims the benefit of U.S. provisional patent applications “Wound Care Image Analysis Using Light Signatures” Ser. No. 63/408,336, filed Sep. 20, 2022, and “Image Analysis Using Skin Wound Factors” Ser. No. 63/451,247, filed Mar. 10, 2023.

The U.S. patent application “Wound Care Image Analysis Using Light Signatures” Ser. No. 18/369,870, filed Sep. 19, 2023, is also a continuation-in-part of U.S. patent application “Multispectral Sample Analysis Using Absorption Signatures” Ser. No. 17/564,318, filed Dec. 29, 2021, which claims the benefit of U.S. provisional patent application “Multispectral Sample Analysis Using Fluorescence Signatures” Ser. No. 63/132,541, filed Dec. 31, 2020.

The U.S. patent application “Multispectral Sample Analysis Using Absorption Signatures” Ser. No. 17/564,318, filed Dec. 29, 2021 is also a continuation-in-part of U.S. patent application “Skin Diagnostics Using Optical Signatures” Ser. No. 17/155,141, filed Jan. 22, 2021, which issued as U.S. Pat. No. 12,053,262 on Aug. 6, 2024, which claims the benefit of U.S. provisional patent applications “Systems and Methods for Wound Care Diagnostics and Treatment” Ser. No. 62/964,969, filed Jan. 23, 2020, and “Multispectral Sample Analysis Using Fluorescence Signatures” Ser. No. 63/132,541, filed Dec. 31, 2020.

Each of the foregoing applications is hereby incorporated by reference in its entirety.

### FIELD OF ART

This application relates generally to image analysis and more particularly to depth-compensated image analysis using multiple light signatures.

### BACKGROUND

A popular family parlor game from the 20th century is known as “Twenty Questions.” It involves having one participant mentally select an item from various categories and having the rest of the participants ask “yes/no” questions to determine the item selected. The rest of the participants get 20 of those yes/no questions, with a successful turn concluding by a participant asking, “Is it X?”, and having “X” indeed be the correct item guessed in 20 questions or less. Typically, a limiting category is provided by the selecting participant from a category list at the beginning of the game. A common category list is “person, place, or thing. Another common category list is “animal, vegetable, or mineral.” Now while it is easy to think of an animal, a vegetable, or a mineral, it is much harder to identify such an item from only a tiny sample that may not look like anything identifiable. While a pig may look like a pig when viewed from a reasonable distance, it may be much harder to ascertain what animal is in view from a single hair sample. A vegetable can be difficult to distinguish with only a small sample. Likewise, many minerals look very similar, but they can have vastly different properties.

Indeed, identifying materials can be a challenging task. An almost infinite number of arrangements of atoms are possible. And some molecules have the exact same atomic composition, but due to different molecular structures, they exhibit vastly different properties. Such molecules, or chemicals, are called isomers. Isomers are molecules that have the same molecular formula but are configured differently. For example, glucose and fructose have the same molecular formula (C₆H₁₂O₆), but they are metabolized by the body differently because of their different configurations. Another example is the molecules formed from C₂H₆O, which can be either dimethyl ether or ethanol. They have the same chemical composition, but different structures. Thus, at room temperature, dimethyl ether is a gas, because it is above its boiling point. However, ethanol is a liquid at room temperature. It can be seen, then, that identification of both chemical composition and chemical structure can be critical.

Organic molecules can exist in extremely complex forms. Organic molecules are found in every part of the human body. Well known examples include DNA, RNA, proteins, organic acids, and carbohydrates, to list just a few. In addition, all life forms include molecules of huge variation and complexity. Identifying organic molecules can be extremely complex. However, identifying which molecules are present in a specimen can be of paramount importance to many fields of endeavor, including chemistry, biology, energy production, forensics, epidemiology, and so on. In fact, identification of molecules found in extraterrestrial samples, such as those obtained from asteroids, moon visits, or other planetary exploration is highly prized and can even be considered as offering clues as to the origins of life itself within the universe.

### SUMMARY

Techniques for depth-compensated image analysis using multiple light signatures are disclosed. The image analysis is based on fluorescence characteristics, reflectance characteristics, and a depth estimation. A material sample is determined to require an analysis of its biophysical status. Biophysical status provides important markers of biomaterial well-being. The biomaterial includes tissue samples. The tissue samples include wounds on a human body that need to be treated medically. The depth-compensated image analysis techniques described herein enable a more accurate and efficacious wound treatment plan. The biomaterial also includes food, drug, and other consumable materials. Multiple light wavelengths are used to illuminate the sample. Multiple light signatures are captured from the sample, based on the fluorescence and reflectance characteristics of the sample. The signatures of the characteristics are more accurately determined from the characteristics by considering the topography of the material sample. Due to inverse square law attenuation of light reflected and/or fluoresced from the material sample, mapping the sample to provide depth-estimated compensation improves accuracy in determining the composition of the material sample, and thus, its biophysical status.

Disclosed techniques describe image analysis using topography-informed excitation and illumination. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

A method for image analysis is disclosed comprising: exciting fluorescence from a material sample, wherein the exciting enables capture of fluorescence characteristics of the material sample, wherein the fluorescence characteristics occur along the Red-Green-Blue (RGB) light wavelength spectrum, and wherein the exciting is accomplished using at least two different fluorescence excitation wavelengths; illuminating the material sample with additional light wavelengths, wherein the illuminating enables capture of reflectance characteristics of the material sample, wherein the material sample exhibits reflectance characteristics along the RGB light wavelength spectrum, and wherein the illuminating is accomplished using at least three additional light wavelengths; mapping a depth estimation of the material sample, based on the reflectance characteristics; and detecting a material composition, wherein the material composition is indicative of a biophysical status of the material sample, and wherein the detecting is based on analysis of the fluorescence characteristics, the reflectance characteristics, and the depth estimation. In embodiments, the mapping a depth estimation is accomplished using a monocular optics system. In embodiments, illumination for the monocular optics system is provided by an integrated smartphone LED. In embodiments, the depth estimation enables inverse square law correction of the fluorescence characteristics and the reflectance characteristics. In embodiments, the inverse square law correction enables depth resolution of three-dimensional (3D) material sample features.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for depth-compensated image analysis using multiple light signatures.

FIG. 2 is a flow diagram for mapping depth estimates.

FIG. 3 is an infographic of machine learning for monocular depth estimation.

FIG. 4 illustrates using a biophysical status.

FIG. 5 is a graph illustrating NAD+ and NADH absorption and emission/fluorescence.

FIG. 6 is a graph illustrating FAD and FADH₂ absorption and emission/fluorescence.

FIG. 7 is a table illustrating detected material sample compositions.

FIG. 8 is a system block diagram for depth-compensated image analysis using multiple light signatures.

FIG. 9 is an infographic for depth-compensated image analysis using a smart device.

FIG. 10 is a system diagram for depth-compensated image analysis using multiple light signatures.

### DETAILED DESCRIPTION

Techniques for depth-compensated image analysis using multiple light signatures are disclosed. The image analysis is based on fluorescence characteristics, reflectance characteristics, and a depth estimation. A material sample is determined to require an analysis of its biophysical status. Biophysical status provides important markers of biomaterial well-being. The biomaterial includes tissue samples. The tissue samples include wounds on a human body that need to be treated medically. The depth-compensated image analysis techniques described herein enable a more accurate and efficacious wound treatment plan. The biomaterial also includes food, drug, and other consumable materials. Multiple light wavelengths are used to illuminate the sample. Multiple light signatures are captured from the sample, based on the fluorescence and reflectance characteristics of the sample. The signatures of the characteristics are more accurately determined from the characteristics by considering the topography of the material sample. Due to inverse square law attenuation of light reflected and/or fluoresced from the material sample, mapping the sample to provide depth-estimated compensation improves accuracy in determining the composition of the material sample, and thus, its biophysical status.

Disclosed techniques include capturing the fluorescence characteristics of a material sample. The fluorescence characteristics are based on light wavelengths emitted by the material sample after excitation by at least two fluorescence excitation wavelengths. The at least two fluorescence excitation wavelengths are provided by one or more controlled light sources capable of emitting such wavelengths. The controlled light sources can emit ultraviolet wavelengths of light. Light along the red-green-blue (RGB) spectrum can extend beyond what is commonly called visible light to longer wavelengths that include what is commonly called infrared light and to shorter wavelengths that include what is commonly called ultraviolet light. The fluorescence characteristics of the material sample are determined in response to the at least two excitation light wavelengths. The light excitation causes molecules of a compound to fluoresce light wavelengths that are generally different from the excitation wavelengths. The fluorescence characteristics can occur in the visible light wavelengths of the RGB spectrum. The total fluorescence characteristics of the material sample in response to the excitation by the at least two fluorescence excitation wavelengths comprise a fluorescence signature.

The light fluoresced by the material sample in response to the excitation wavelengths (and also the light reflected by the material sample in response to the illumination wavelengths, described below) can be detected by an RGB-imaging sensor. The RGB image sensor can be a standalone sensor, a sensor integrated in a color camera, a sensor integrated in a custom imaging device, a sensor normally included in a smartphone, and so on. The RGB image sensor can be implemented using one or more RGB sensors. The RGB sensors can include discrete and/or integrated filters. The RGB sensors can employ various optical lenses. The RGB sensors can include various electronic controls and processors that are employed in their image capturing. The RGB sensors can be components of a broad-spectrum image sensor. The broad-spectrum image sensor can employ an integrated, very low-cost Bayer filter which enables the broad-spectrum image sensor to provide sensitivities to particular wavelengths, including light from frequencies which are visible to the human eye and those which are not. Various materials fluoresce and reflect different wavelengths of light when compared to one another. However, imaging such as multispectral imaging can be used to differentiate materials based on their spectral fluorescence signatures and characteristics and their spectral reflectance/absorption signatures and characteristics. The depth-compensated image analysis using multiple light signatures techniques that are disclosed herein can reduce the complexity, cost, and deployment challenges when compared to using specialized multispectral cameras, elaborate optical filters, and expensive filter wheels. For example, filter wheels can have significant orientation and alignment sensitivities. By contrast, the multispectral analysis described herein can be performed without employing fixed, lab-only equipment placement.

Disclosed techniques include capturing the reflectance characteristics of the material sample. The reflectance characteristics are based on illuminating a material sample with at least three additional light wavelengths to determine absorption characteristics of the material sample. The additional light wavelengths can include an additional visible light wavelength, an IR wavelength, an NIR wavelength, a UV wavelength, and the like. The additional light wavelengths are used to illuminate the material sample, and then the reflected light is measured to provide the sample's reflectance characteristics. The total reflectance characteristics of the material sample in response to the illumination by the at least three additional light wavelengths comprise a reflectance signature. The reflectance response can determine whether a light wavelength is absorbed by the material sample, and if so, how much of the light wavelength is absorbed. The amount of light that is absorbed can be determined by capturing an amount of light reflected by the material sample and referencing that amount to the amount of light illuminating the material sample. Compensation can be included to cancel any ambient lighting contributions. The compensating can include enhancing the signature, normalizing the signature, augmenting the signature, subtracting the ambient light signature, etc. A further, significant compensation is described below.

Disclosed techniques include mapping a depth estimation of the material sample. The abovementioned signatures of the fluorescence characteristics and the reflectance characteristics are more accurately determined from the characteristics by considering the topography of the material sample. Due to inverse square law attenuation of light reflected and/or fluoresced from the material sample, mapping the sample to provide depth-estimated compensation improves accuracy in determining the composition of the material sample, and thus, its biophysical status. Because mapping depth normally requires some kind of stereoscopic optics system, which is not typically available in a low-cost imaging system, such as the imaging system of a smartphone, techniques are disclosed to adapt a monocular optics system using a “white” light source and machine learning to estimate the depth of each material sample pixel captured by the RGB sensor. A low cost time-of-flight sensor may be used to augment and improve the accuracy of the monocular depth estimation. The response of the material sample to the impinging excitation light wavelengths and illumination light wavelengths can be captured by measuring the output values of one or more RGB image sensors on a pixel-by-pixel basis. The output values can then be adjusted, or compensated, by the depth estimation mapping before being analyzed. The analysis enables a composition of the material sample to be detected. The detected composition can be indicative of a biophysical status of the material sample.

Note that RGB image sensors generally demonstrate peak blue sensitivity at 400 nm to 475 nm, peak green sensitivity at 475 nm to 580 nm, and peak red sensitivity at 580 nm to 750 nm. Excitation light sources at wavelengths near the edge of, inside of, or outside of the RGB visible light wavelength spectrum, which can be referred to as the extended RGB spectrum, can include wavelengths from 350 nm to 950 nm, for example. However, note that the definition of the exact wavelengths of visible light is somewhat subjective. For purposes of discussion, a visible light wavelength spectrum of about 425 nm to 725 nm is understood herein, although discrete wavelengths or wavelength ranges are used herein when possible. Also note that due to manufacturing and design tolerances, while a given component may be specified for a given wavelength or wavelength range emission or reception, the wavelengths disclosed herein represent actual, real-life componentry with practical limitations. Hence, when a wavelength is described as substantially at a certain value, it is to be understood that a 5 nm to 10 nm tolerance is appropriate.

The RGB sensor can detect multispectral fluorescence responses of a material to various wavelengths of light. The RGB sensor typically is mass produced and has applications in low-cost technology that endeavors to detect light waves in the visible spectrum in a standard three-color, RGB palette suitable for digital processing. The RGB sensor typically employs an integrated Bayer filter applied during the manufacturing process of a CMOS, CCD, or similar sensor semiconductor fabrication. The Bayer filter is completely integrated within or to the sensor and cannot be removed, replaced, or adjusted. When light impinges the surface of an RGB sensor, the underlying photosensors register a signal related to the intensity of the impinging wavelengths as a function of the color of the integrated filter directly over each photosensor device. The disclosed technology does not require expensive special cameras, filter wheels, complex optical alignments, or stationary, non-handheld components.

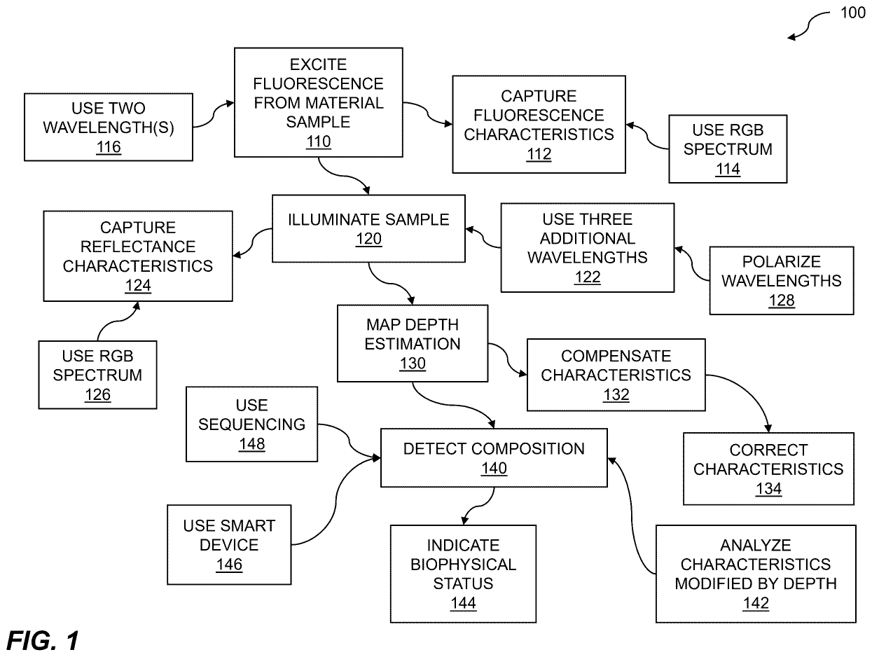

FIG. 1 is a flow diagram for depth-compensated image analysis using multiple light signatures. Biophysical status of a material sample such as a skin wound can be based on topography-informed excitation and illumination. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

The flow 100 includes exciting fluorescence from a material sample 110. The material sample can include biomaterials, drugs, skin wounds, foods, wound exudate, and so on. The material sample can require a biophysical status. The biophysical status can be indicated by the composition of the material sample. For example, in a clinical setting, a doctor may try to determine how a patient's wound is progressing toward healing. However, the naked eye can only provide a certain amount of information, and it certainly cannot determine the presence and concentration of individual chemicals, biochromes, analytes, etc., which can be critical to the determination. The fluorescence that is excited from the material sample can be recorded by an image sensor and can enable capturing of the fluorescence characteristics 112 of the material sample. The fluorescence characteristics can occur and be captured using the RGB spectrum 114. The RGB spectrum can be sensed by RGB image sensors, such as the image sensor (“camera”) integrated in many smartphones. The exciting can be accomplished using at least two different fluorescence excitation wavelengths 116. A fluorescence excitation light wavelength signal can have a wavelength which is less than a wavelength of the RGB light wavelength spectrum. The wavelength less than a wavelength of the RGB light wavelength spectrum can be substantially between 200 nm and 425 nm. The wavelength bands can include a first band of the optical excitation light wavelength bands comprising wavelengths substantially in the range of 325 nm to 375 nm and a second band of the optical excitation light wavelength bands comprising wavelengths substantially in the range of 375 nm to 425 nm. In embodiments, the two different fluorescence excitation wavelengths comprise substantially a 365 nm wavelength and substantially a 405 nm wavelength.

The flow 100 includes illuminating the material sample 120 with at least three different additional light wavelengths 122 (i.e., additional beyond the at least two different fluorescence excitation wavelengths). The illuminating enables capturing reflectance characteristics 124 of the material sample. The reflectance characteristics can be captured with an RGB image sensor by using the RGB light wavelength spectrum 126. The three additional light wavelengths can include far-infrared (FIR), mid-infrared (MIR), and near-infrared (NIR), visible light, and so on. In embodiments, the at least three additional light wavelengths can include a blue-band light wavelength, a green-band light wavelength, and a red-band light wavelength. The blue-band light, the green-band light, and the red-band light can include various wavelengths of blue, green, and red light, respectively. The illuminating can be used to scan a plurality of optical excitation visible light wavelength bands on a material sample. The material sample can exhibit optical spectral reflectance characteristics along the RGB light wavelength spectrum. The material sample can comprise one or more materials, tissues, wounds, wound exudate, biologics, drugs, foods, agricultural products, and so on. The optical excitation light wavelength bands can be provided by various sources including an incandescent light source, an LED light source, a laser light source, and so on. The light sources can emit narrow spectra of light at various peak wavelengths across the visible light (RGB) spectrum. The illumination wavelengths can be targeted toward material sample reflectance. A reflectance excitation light wavelength signal can have a wavelength contained within the wavelengths of the RGB light wavelength spectrum, i.e., approximately 400 nm to approximately 700 nm. Note that there are varying definitions of the exact wavelengths comprising the RGB, or visible light, spectrum. However, whatever those definitions are, they do not override the wavelengths described in the techniques herein. A first illumination wavelength can be substantially 475 nm. A second illumination wavelength can be substantially 530 nm. A third illumination wavelength can be substantially 665 nm. Additional illumination wavelengths can also be used.

The flow 100 includes polarizing the three illumination wavelengths 128, relative to the RGB image sensor polarization. The polarization can be a perpendicular polarization with respect to the RGB image sensor. As discussed above and throughout, the RGB image sensor can comprise the integrated camera of a smartphone. For example, two polarizers that are oriented perpendicular to one another can be situated, one in front of a smartphone camera (RGB sensor), polarizing along one axis, and one in front of reflectance light emitting diodes (LEDs), polarizing along a perpendicular axis. This polarization can eliminate specular reflection from the received image and can increase the accuracy of the reflectance data. A clever element of this approach is that a specular reflection is obtained from the integrated smartphone LED, because it does not sit behind a polarizer. Note that the integrated smartphone LED, typically used for smartphone flashlight and camera flash purposes, is not polarized, which can preserve specular reflections, which can be important for the depth estimation mapping, discussed below.

The flow 100 includes mapping a depth estimation 130. The mapping can provide a depth estimation across the x-y image of the material sample. The depth estimation describes the topography of the material sample and can be used for compensating characteristics 132 for inverse square law losses of light energy that are captured. Because light travels in three dimensions, its attenuation is proportional to the square of the distance between source and sample and sample to image sensor. The compensation can be for correcting the characteristics 134 for the light energy lost. For example, a skin wound may have a ridge around the outside of the wound and a hole in the center of the wound. Although this topographical difference may only be one or two centimeters, it can be significant enough to distort the detection of the material composition of the sample. The mapping can be accomplished using a monocular optics system. The depth estimation can be accomplished using a machine learning model. The depth estimation can be performed by illuminating the material sample with a white-light LED, such as the flashlight or camera flash LED integrated in most smartphones. The mapping can be used to more accurately analyze the fluorescence characteristics and the reflectance characteristic of the material sample, based on the compensating. The monocular depth estimation can be supplemented and/or augmented by a single-point time of flight sensor, such as a single-point lidar sensor. The lidar sensor can be integrated in the smartphone or provided separately, for example, packaged with the LED sources for the illumination wavelengths.

The flow 100 includes detecting the composition 140 of the material sample. The detecting is based on analyzing the characteristics modified by the depth mapping 142. The material composition can be detected by noting the topologically-aware peaks, valleys, and relative intensities of the depth-corrected fluorescence characteristics and reflectance characteristics which are responsive to the impinging amplitude and wavelengths of the excitation light and the illumination light. The depth-corrected fluorescence characteristics and reflectance characteristics can be based on using sequencing 148 of the at least two fluorescence excitation wavelengths and the at least three additional light wavelengths. Sequencing can provide less “crosstalk” between impinging excitations and illuminations. In addition, an image can be captured while all excitation wavelengths and all illumination wavelengths are off, which can enable “zeroing out” the effects of ambient light. Other techniques for ignoring ambient light effects can include using a formal or makeshift light hood or even turning off all or most of a room's lighting, including closing blinds to remove natural light effects. The detecting can be accomplished using a smart device 146. The smart device can include a smartphone, a tablet, a phablet, or other commercially available, inexpensive device with a suitable integrated image sensor. In addition, the camera LED flash/smartphone flashlight can be used to provide the predominantly white light that can be used for mapping the depth estimation.

The composition of the material sample can be used for indicating the biophysical status 150 of the material sample. The biophysical status of a material sample, such as a sample obtained from a skin wound, an in situ skin wound, and so on, can be used to gauge, ascertain, track, and so on a status of the skin wound. The status can be based on the presence or absence of one or more analytes, the presence or absence of infection, and so on. The biophysical status can be based on the presence or absence of cells within skin wound exudate. In embodiments, the biophysical status can enable a skin wound assessment. The skin wound assessment can be critical to determining a stage of wound healing, the efficacy of a wound treatment plan, and the like. Such skin wound assessment can be particularly critical in tracking the healing of chronic wounds. The chronic wounds can result from diseases such as diabetes or conditions such as venous or arterial insufficiency. In embodiments, the skin wound assessment can be performed longitudinally. The longitudinal assessment can include skin wound assessment over a period of time. The period of time can include one or more hours, days, weeks, months, etc. In embodiments, the skin assessment performed longitudinally can enable a wound care treatment plan. The treatment plan can include a drug therapy, bandage type and bandage change frequency, surgery, etc. In other embodiments, the skin assessment performed longitudinally can enable development of a wound healing trajectory. A wound healing trajectory can be based on stages of healing. The stages of wound healing associated with a healing trajectory can include stages such as hemostasis, inflammatory, proliferation, and maturation stages. In further embodiments, the wound healing trajectory can be used to modify a wound care treatment plan.

The detecting the composition 140 can be based on analysis of images captured by an imaging device, such as the camera of a smartphone or other RGB image capture sensor. The images that are captured can include the fluorescence characteristics and the reflectance characteristics of the material sample. The fluorescence characteristics and the reflectance characteristics are compensated by the depth estimation mapping to allow corrected intensity values for the characteristics. The characteristics provide a signature for various substances present in the material sample, such as biologic chromophores that may be present in a skin wound sample. The composition can be indicative of a biophysical status of the material sample, such as water-rich or water-starved. The signatures can be analyzed on a per impinging light wavelength basis. Signatures for the at least two excitation wavelengths can be based on a first wavelength of the at least two excitation wavelengths, a second wavelength of the at least two excitation wavelengths, a combination of both the first and second wavelengths of the at least two excitation wavelengths, and so on. Similar sequencing and signature definition can be used for the at least three additional illumination wavelengths. For example, for protoporphyrin, which can be a precursor to heme, the signature would include a high intensity signal when excited at 405 nm and an absence of signal when excited at 365 nm. Other responses, described later, can be included in a catalog of responses that are compared to a captured signature as part of the analysis. In embodiments, the analysis can include identifying infection within the material sample. An infection can be based on the presence of undesirable bacteria, fungi, microorganisms, viruses, yeast, etc. An infection can first occur in a portion of a body and can spread to another portion of the body. An infection can include a chronic infection. The infection can destroy tissue thereby hindering wound healing, worsening a wound status, etc. An infection can present as an elevated body temperature, suppuration, etc. In embodiments, the infection can be identified based on a measure of a porphyrin, pyoverdine, slough, eschar, or an inflammation signature. The measure of porphyrin, pyoverdine, slough, eschar, or an inflammation signature can be determined based on of the fluorescence characteristics and the reflectance characteristics of the skin wound sample, by examining the wound exudate, etc. In embodiments, the inflammation signature can include wound temperature and wound water content. The wound temperature can be based on capturing a thermal image of the skin wound. The wound water content can be determined by analyzing the fluorescence characteristics and the reflectance characteristics of the skin wound. In embodiments, identifying wound topology, inflammation, epithelialization, granulation, and infection comprises a five-factor biophysical material sample status.

Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

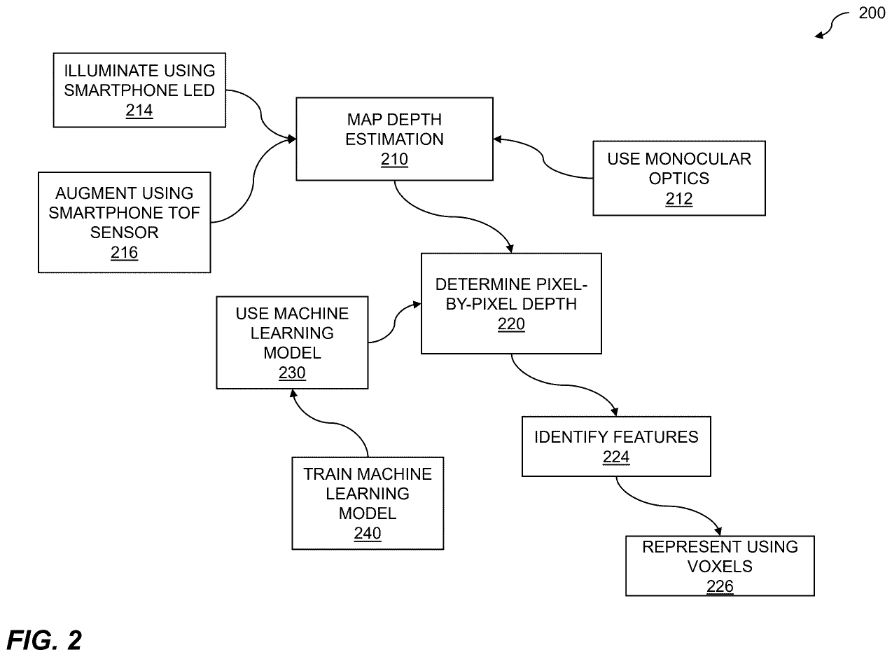

FIG. 2 is a flow diagram for mapping depth estimates. Depth estimates of a material sample can be based on topography-informed excitation and illumination. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

The flow 200 includes mapping a depth estimation 210. As discussed above, topological variation in the material sample can result in skewed results due to varying inverse square law losses of the light wavelength energies being captured. The depth estimation uses monocular optics 212, rather than expensive stereoscopic cameras or another complicated approach. The monocular optics can be provided by a smart device, using the built-in imaging system readily available in many smart device commercial offerings. The monocular optics-based mapping can be illuminated using the normal, integrated smartphone flash LED 214. A smartphone flash LED can provide broad spectrum, white light, which can enable the depth estimation. The flow 200 includes augmenting the depth estimation using an integrated, smartphone time-of-flight (TOF) sensor 216. A TOF sensor, such as a built-in light detection and ranging (LIDAR) sensor, can be included as an integrated part of a smartphone and repurposed from its normal photo-enhancing use to provide the range (distance) and shape of the material sample. Data from the TOF sensor can further be used to calibrate the monocular depth estimation mapping.

The flow 200 includes determining a pixel-by-pixel depth 220 of the material sample. The pixel-by-pixel depth enables identifying features 224 of the material sample. The features identified by analyzing the pixel-be-pixel depth can include wound features, such as ridges around the outside of a wound, depression(s) in the interior of the wound, bumps across the surface of the wound, and so on. Such features are identified in three dimensions (3D). The 3D identification can be represented using voxels 226. A voxel can include a depth parameter in association with a corresponding pixel datum. Various data structures can be employed for voxel description and manipulation. In embodiments, the mapping a depth estimation is accomplished using a monocular optics system. In embodiments, illumination for the monocular optics system is provided by an integrated smartphone LED. In embodiments, the depth estimation enables inverse square law correction of the fluorescence characteristics and the reflectance characteristics. In embodiments, the inverse square law correction enables depth resolution of three-dimensional (3D) material sample topological features. In embodiments, the 3D material sample features include tissue topology. In embodiments, the tissue topology is captured by voxels. Some embodiments comprise using a time-of-flight sensor to calibrate the depth estimation.

The flow 200 includes using a machine learning model 230 to determine the pixel-by-pixel depth mapping. The machine learning model can comprise a convolutional neural network, a deep neural network, a recurrent neural network, a support vector machine, and so on. The machine learning model can enable analysis of a two-dimensional (2D) image to infer a third, depth dimension representative of the topography of the material sample. The inference can be performed on images captured by a simple, monocular optics system for capturing 2D images. The flow 200 includes training the machine learning model 240. The training can be based on obtaining 3D images of material samples with various topographies and comparing them with 2D images of the same topography. The 3D images can be obtained using a depth-capable camera, such as a stereoscopic camera. The 2D images can be obtained using a variety of commercially available monocular-optics imagers, such as those found in many smartphones. In embodiments, the depth estimation is accomplished using a machine learning model. In embodiments, the machine learning model is trained using monocular RGB images compared with depth-enabled images. In embodiments, depth enabled images are captured with a depth-capable camera.

Various steps in the flow 200 may be changed in order, repeated, omitted, or the like without departing from the disclosed concepts. Various embodiments of the flow 200 can be included in a computer program product embodied in a non-transitory computer readable medium that includes code executable by one or more processors.

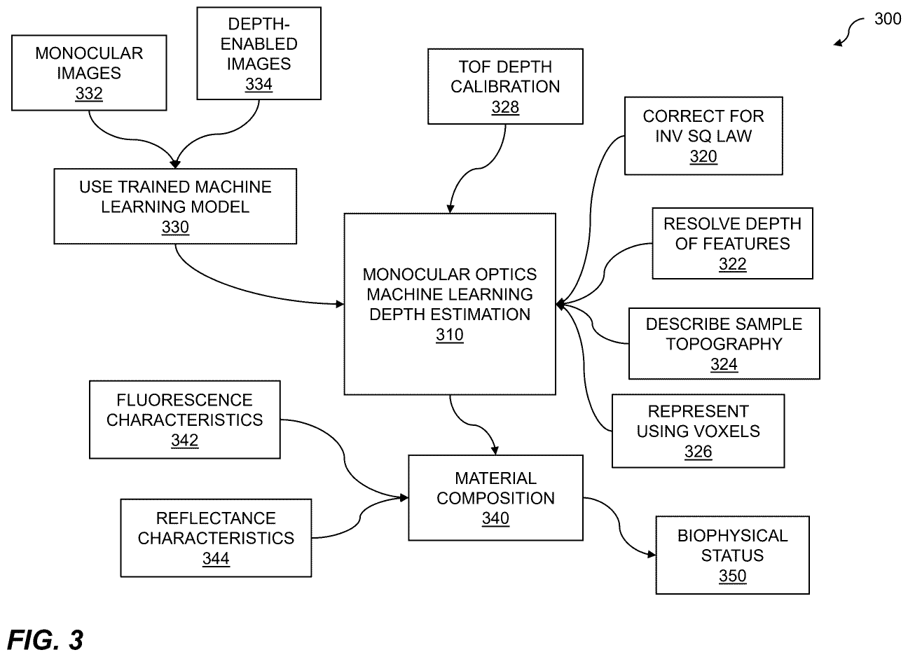

FIG. 3 is an infographic of machine learning for monocular depth estimation. Machine learning can enable depth-compensated image analysis using multiple light signatures. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

The infographic 300 describes monocular optics machine learning depth estimation 310, which can enable image-based detection of material composition. As described above and throughout, the depth estimation includes correcting for inverse square law losses 320 due to variation in material sample topography, resolving the depth of features 322 that describe the material sample topography 324, and representing the topography using voxels 326. The monocular depth algorithm can estimate relative depths, but in addition, it can be augmented by an absolute depth measurement from a single-point time-of-flight (TOF) module. The monocular depth algorithm can be used in combination with data from the TOF module by measuring the area of the material sample that the TOF module surveys and then normalizing the monocular depth map to match the reading of that area. In embodiments, a time-of-flight module is used to calibrate the depth estimation.

The infographic 300 includes monocular optics-based depth estimation performed using a machine learning model that has been trained 330 and calibrated 328. The training dataset is developed by taking depth-enabled images 334 and comparing them to one or more corresponding monocular images 332. The monocular images can be taken by a variety of monocular optics-based imaging systems, including commercially available smartphone imaging (camera) systems. In the infographic 300, the monocular optics machine learning depth estimation 310 is important in determining a material composition 340. The depth estimation provides a mapping of the material sample that is used to compensate for distance variation and correct fluorescence characteristics 342 and reflectance characteristics 344 that have been captured by a monocular-optics imaging system. The corrected characteristics represent signatures indicative of a biophysical status 350 of the material composition.

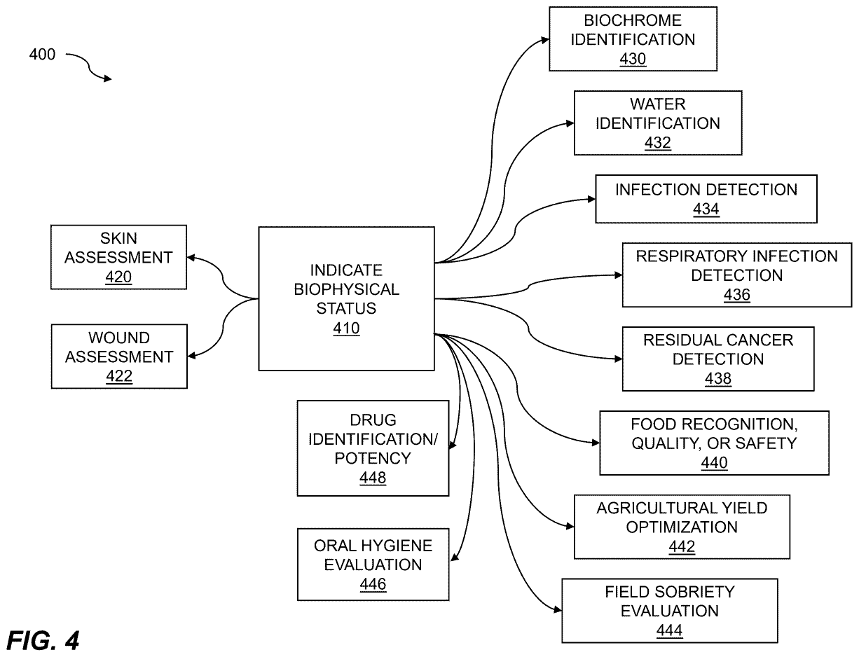

FIG. 4 illustrates using a biophysical status. A biophysical status can be the result of depth-compensated image analysis using multiple light signatures. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

Various types of material samples can be analyzed. In embodiments, the material sample can include a wound topology. An indication of biophysical status associated with material sample composition can be useful to a variety of human endeavors, as will be discussed below. The illustration 400 includes generating an indication of biophysical status 410 of a material sample. As discussed throughout, at least two light wavelengths are excited on a material sample. The exciting enables capture of fluorescence characteristics of the material sample. The material sample exhibits fluorescence characteristics along the Red-Green-Blue (RGB) light wavelength spectrum. The material sample is illuminated with at least three additional light wavelengths. The illuminating enables capture of reflectance characteristics of the material sample. The material sample exhibits reflectance characteristics along the Red-Green-Blue (RGB) light wavelength spectrum. An output indicative of biophysical status of the material sample is generated. The output is based on analysis of the fluorescence characteristics and the reflectance characteristics. A thermal image of the material sample is further captured. The output is augmented based on an analysis of the thermal image.

The generated indication of biophysical status of the material sample can enable a skin assessment 420. The skin assessment can involve predicting the onset of skin conditions such as psoriasis, which can be distinguished based on fluorescence from fluorophores such as melanin, elastin, collagen, keratin, and flavoprotein. Other skin conditions, such as eczema and acne, can also be predicted. In addition, skin hydration can be assessed using the disclosed techniques. The skin assessment can include feature identification. The indication can enable a wound assessment 422. The wound assessment can be based on collecting a variety of images at different excitation wavelengths and spatially registering the images using micro- or macro-scale features, skin and wound edges, fiducial marks, reference standards for alignment, corresponding biological features, and the like. Feature recognition can be accomplished using Laplace of Gaussians, difference of Gaussians, Hessian-Laplace, scale invariant feature transform (SIFT), multi-scale-oriented patches (MOPS), or other image processing techniques for local feature description. Once corresponding features on images are identified, the registration technique can use translation, rigid body, rotation, or affine transformation methods to register multiple images collected at different wavelengths. A pixel-by-pixel registration allows for the images to be digitally processed in order to identify biological features, to perform calculations which isolate or enhance the biological signals, and/or to assess wound healing. Further analysis can enable algorithmic identification of infection. In embodiments, the wound assessment includes infection detection. In embodiments, the skin assessment includes wound assessment. In embodiments, the wound assessment is taken over time. In embodiments, the wound assessment is repeated over time and enables a wound care treatment plan. In embodiments, the skin assessment is updated using temporal change feature matching, that is, by comparing identified features in the wound to determine how they are changing temporally (i.e., with the passage of time). The temporal change can occur over two or more healthcare clinical sessions. At least one of the two or more healthcare clinical sessions can be self-administered.

As discussed previously, the indication can enable biochrome identification 430 and water identification 432. In addition, the indication can enable infection detection 434 or respiratory infection detection 436. Host metabolism plays a vital role in viral infections. Energy yielding metabolic pathways are repurposed by the virus to support viral replication. High concentrations of nicotinamide adenine dinucleotide+hydrogen (NADH) and flavins are indicative of such infections. The indication can be generated by isolating signals from NADH and flavins by collecting fluorescence photons in the R, G, and B channels, respectively, and exciting at or near 400 nm. This approach further isolates features in an image that can be attributed to the presence of flavins and NADH by taking the normalized ratio, where normalization is based on excitation flux, integration time, and channel sensitivity of the green channel signal to the blue channel signal and isolating based on pixels that yield a ratio value indicative of the presence of NADH and/or flavins.

In addition, abnormal concentrations of porphyrin, which can be detected using the disclosed concepts, have been observed in serum from COVID-19 patients. Other respiratory-related infections, such as sinusitis, are more prevalent with a common cold than with influenza. These infections can be analyzed based on the fact that signatures of sinusitis, such as fluid in the sinuses, can increase the indication precision to distinguish between respiratory infection types. Furthermore, common cold viruses usually do not cause substantial damage to the airway epithelium, whereas influenza and COVID-19 can damage cells in the respiratory epithelium. In fact, a broad variety of respiratory pathogens, including rhinoviruses, coronaviruses, and the like, can adversely affect cells. Redness and inflammation associated with such cellular damage can be detected using the disclosed techniques. By applying the disclosed techniques when looking into a patient's throat and taking images to measure fluorescence, absorption, and thermal radiation from the throats of patients with possible infection from respiratory viruses such as SARS-COV-2, Influenza A, and Influenza B, infection can be detected. Such methods can also facilitate telemedicine diagnostics. In embodiments, the indication enables infection detection. In embodiments, the infection detection is based on biochrome identification. In embodiments, the indication enables respiratory infection detection. In embodiments, the respiratory infection detection comprises influenza detection. In embodiments, the influenza detection comprises COVID-19 detection.

This technology isolates signals from infection-associated biochromes, such as porphyrin and pyoverdine, by holding an excitation wavelength constant and collecting signals from progressively longer wavelength emission channels. This action is performed at each pixel in an image. In one embodiment, fluorescence is collected by exciting wavelengths in the blue/UV region of the spectrum such that the peak of the spectral distribution of the excitation source is at a lower wavelength (higher energy) than what is typically detected by the sensor (CMOS or CCD as examples) that is being used for detecting photons and generating an image.

The indication can enable residual cancer detection 438. Autofluorescence imaging is enabled by the disclosed concepts and has been used to diagnose oral cancer, breast cancer, lung cancer, skin cancer, brain cancer, and others. Autofluorescence from NADH has been cited as one possible biomarker for targeting cancer. Similarly, fluorescence from dense connective tissue (extracellular matrix, etc.) associated with a tumor can be used to delineate tumor boundaries. In addition, such techniques can enable detection of residual cancer during surgery. In embodiments, the indication enables residual cancer detection. In further embodiments, the residual cancer detection occurs during oncological surgery.

The indication can further enable food recognition, food quality, or food safety 440. Common foodborne pathogens include E. coli, Salmonella, Listeria, Cyclospora, and Hepatitis A. Disclosed techniques can enable rapid detection of foodborne pathogens in order to avoid distribution of contaminated foods. Authentication, quality, and possible adulteration of food must be monitored for distribution and consumption. For example, liquor, wine, and beer inspection can be performed by analyzing both water content and the presence of fluorescent compounds. Fluorescent compounds such as polyphenols, flavonoids, stilbenes, tannins, coumarins, and fluorescent amino acids are key markers of authenticity and quality. In some embodiments, two or three excitation LEDs at different blue and UV wavelengths may be employed for determining a shift in emission resulting from a change in excitation frequency. Such techniques can be used in plant food quality analysis, milk quality analysis, fruit quality analysis, coffee quality analysis, as well as protein quality analysis of products as varied as beef and sashimi, to name just a few. Other applications include monitoring the progress of fermentation, such as malolactic fermentation, for the deacidification of red wines. In-line monitoring of the fermentation process can also be applied to fermentation processes in which yeast or bacteria are programmed to produce a specific chemical such as THC and CBD. In addition, monitoring caloric intake can be enabled by food composition and rough, overall portion size identification. In embodiments, the indication of composition of the material sample includes identification of the presence of water, and the presence of water is used to determine organism health for the material sample. In embodiments, the indication enables food recognition, food quality, or food safety identification. In embodiments, the food quality detects food adulteration. And in embodiments, the food quality monitors progression of fermentation.

The indication can enable agricultural yield optimization 442. Especially in automated indoor farming, which is poised to assume a significant burden of the food supply, the disclosed techniques can enable identification of crop ripeness, crop water sufficiency, crop fertilization sufficiency, crop disease detection, and so on. This approach can enable minimized use of insecticides and herbicides while optimizing crop yield. In addition, a robot- or drone-based approach to agricultural optimization is feasible due to the portable attributes of the disclosed techniques. In embodiments, the indication enables agricultural yield optimization. In embodiments, providing excitation and measuring RGB sensor output values are accomplished using drone technology. The indication can have applications in law enforcement and can enable a field sobriety evaluation 444 for an individual. A contactless evaluation using the disclosed techniques can determine the need for a more invasive breathalyzer test. In addition to visual indicators such as enlarged pupils and eye movement that is faster than normal, measured amounts of vasoconstriction and vasodilation, depending on a level of intoxication, can be enabled using the indication. In embodiments, the indication enables field sobriety evaluation of individuals. In embodiments, the field sobriety evaluation of individuals is accomplished in a contactless manner. The indication can have further applications in dental care. The indication can enable an oral hygiene evaluation 446 for an individual. This can include detecting plaque, gingivitis, and other dental abnormalities using multispectral imaging and fluorescence. Thus, in embodiments, the indication enables oral hygiene evaluation. The indication can have further applications in drug identification and potency 448. Drug identification and drug potency evaluation are critical for many applications, particularly in managing illegal drugs, especially those illegal drugs laced with unsuspected contaminants, such as fentanyl. Rapid and accurate evaluation of material samples with suspected illegal drug content can be a critical component in saving lives. In embodiments, the material sample comprises a tissue sample. In embodiments, the tissue sample includes a wound. In embodiments, the exciting, the illuminating, the mapping, and the detecting determine a wound healing metric. In embodiments, the material sample comprises an edible biomaterial.

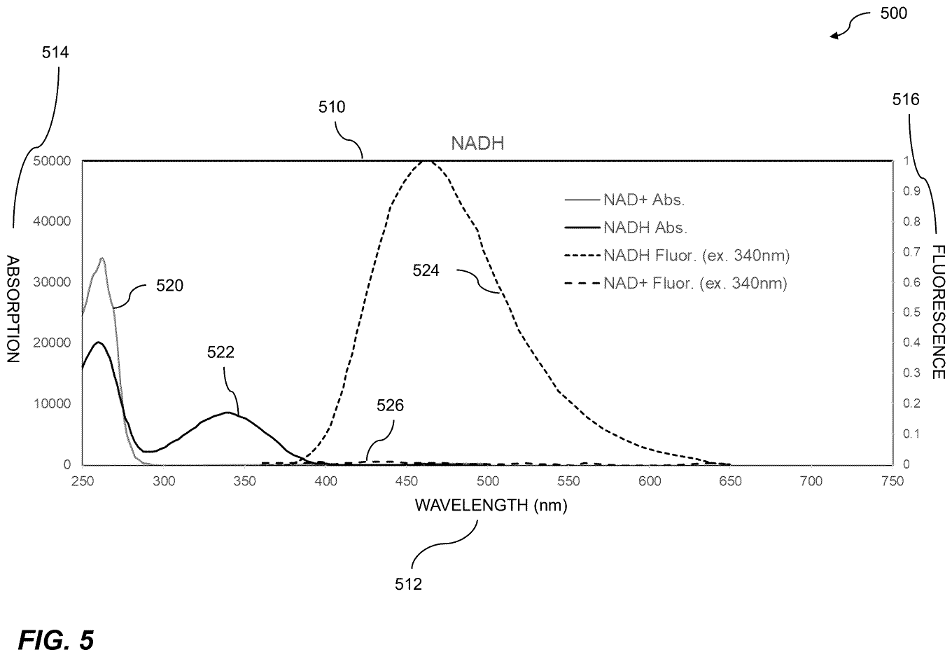

FIG. 5 is a graph illustrating NAD and NADH absorption and emission/fluorescence. NAD⁺ and NADH absorption and emission/fluorescence can be captured and used in depth-compensated image analysis using multiple light signatures. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

In the illustration 500, a graph 510 is shown that illustrates oxidized nicotinamide adenine dinucleotide (NAD) and reduced nicotinamide adenine dinucleotide (NADH) absorption and emission/fluorescence around a wound. The graph 510 shows an x-axis of wavelengths in nanometers 512, a left y-axis of absorption intensity in arbitrary units 514, and a right y-axis of relative fluorescence 516. Note that a light wavelength such as an ultraviolet light emission at a certain wavelength can cause fluorescence at a different wavelength by the wound sample. The excitation response wavelengths based on the plurality of excitation wavelengths can include a fluorescence signature for the wound sample. An absorption signature for a substance of interest in the wound sample can be determined using spectrophotometry. The wound sample can be analyzed to provide a metric of wound monitoring. In embodiments, the metric of wound healing can include an epithelialization metric. The epithelialization metric can be based on detecting one or more factors around the wound from which the wound sample was obtained. The factors can be obtained by detecting NAD⁺, NADH, FAD, and FADH₂ around the wound. Ratios can include a ratio of NAD⁺ to NADH and/or FAD to FADH₂ around the wound. The area around the wound can include wound edges, wound perimeters, wound centers, wound areas, periwound regions, and so on.

The graph 510 includes absorption signatures for NAD⁺ 520 and NADH 522. It can be seen that while NAD 520 and NADH 522 have absorption peaks around 270 nm, NADH also has a localized peak at around 340 nm. Using 340 nm as an excitation wavelength produces a large fluorescence response peak for NADH 524 at about 460 nm, while the NAD″ fluorescence response 526 exhibits almost no fluorescence. The optical excitation ultraviolet wavelength band(s) can be chosen for detection of NAD and/or NADH. The choices of optical excitation ultraviolet wavelength bands can be based on emitter cost, availability, commercial availability versus custom availability, and so on. In embodiments, the NAD and/or NADH optical excitation ultraviolet light wavelength bands can include wavelengths substantially in the range of 325 nm to 400 nm. One or more wavelengths can be provided. In a usage example, the optical excitation ultraviolet wavelength can include substantially a 340 nm excitation wavelength. Other wavelengths may be used, however, such as a substantially 365 nm wavelength, which may be a wavelength whose emitter is readily available, inexpensively available, useful for other biochromes of interest, and so on. An additional optical excitation ultraviolet light wavelength can be present. In the case of considering a ratio of NAD⁺ to NADH fluorescence, the fluorescence from NADH excited by a 340 nm wavelength is ˜100× larger than the NAD fluorescence. Therefore, measuring a fluorescence signal at 460 nm after excitation at 340 nm can be a useful indicator of metabolic state.

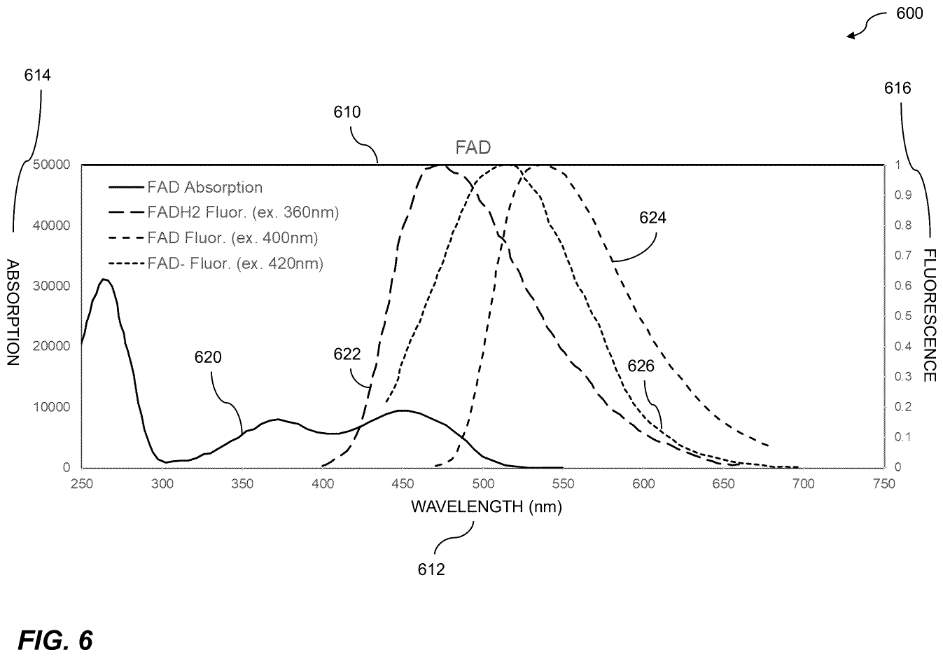

FIG. 6 is a graph illustrating FAD and FADH₂ absorption and emission/fluorescence. FAD and FADH₂ absorption and emission/fluorescence can be captured and used in depth-compensated image analysis using multiple light signatures. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

In the illustration 600, a graph 610 is shown that illustrates oxidized flavin adenine dinucleotide (FAD) and reduced flavin adenine dinucleotide (FADH₂) absorption and emission/fluorescence around a wound. The graph 610 shows an x-axis of wavelengths in nanometers 612, a left y-axis of absorption intensity in arbitrary units 614, and a right y-axis of relative fluorescence 616. Note that a light wavelength such as an ultraviolet light emission at a certain wavelength can cause fluorescence at a different wavelength by the wound sample. The excitation response wavelengths based on the plurality of excitation wavelengths can include a fluorescence signature for the wound sample. An absorption signature for a substance of interest in the wound sample can be determined using spectrophotometry. The wound sample can be analyzed to provide a metric of wound monitoring. In embodiments, the metric of wound healing can include an epithelialization metric. The epithelialization metric can be based on detecting one or more factors around the wound from which the wound sample was obtained. The factors can be obtained by detecting NAD⁺, NADH, FAD, and FADH₂ around the wound. Ratios can include a ratio of NAD⁺ to NADH and/or FAD to FADH₂ around the wound. The area around the wound can include wound edges, wound perimeters, wound centers, wound areas, periwound regions, and so on.

The graph 610 includes an absorption signature for FAD 620. It can be seen that while FAD 620 has an absorption peak around 270 nm, FAD also has localized peaks at around 370 nm and 450 nm. Using 400 nm as an excitation wavelength produces a large fluorescence response peak for FAD 624 at about 540 nm, while using 420 nm as an excitation wavelength produces a large fluorescence response peak for FAD 626 at about 520 nm. Furthermore, using 360 nm as an excitation wavelength produces a large fluorescence response peak for FADH₂ 622 at about 470 nm. The optical excitation ultraviolet wavelength band(s) can be chosen for optimized detection of FAD and/or FADH₂ and/or NAD and/or NADH. The choices of optical excitation ultraviolet wavelength bands can be based on emitter cost, availability, commercial availability versus custom availability, and so on. In embodiments, the FAD and/or FADH₂ optical excitation ultraviolet light wavelength bands can include wavelengths substantially in the range of 325 nm to 400 nm. One or more wavelengths can be provided. In a usage example, the optical excitation ultraviolet wavelength can include substantially a 360 nm excitation wavelength. Other wavelengths may be used, however, which may be a wavelength whose emitter is readily available, inexpensively available, useful for other biochromes of interest, able to be combined for detection of other biochromes, and so on. An additional optical excitation ultraviolet light wavelength can be present. In the case of considering a ratio of FAD to FADH₂ fluorescence, the fluorescence from FAD excited by a 400 nm wavelength produces a very different peak from the fluorescence of FADH₂ excited by a 360 nm wavelength (540 nm vs. 470 nm). Therefore, measuring a fluorescence signal at 470 nm after excitation at 360 nm can be a useful indicator of metabolic state.

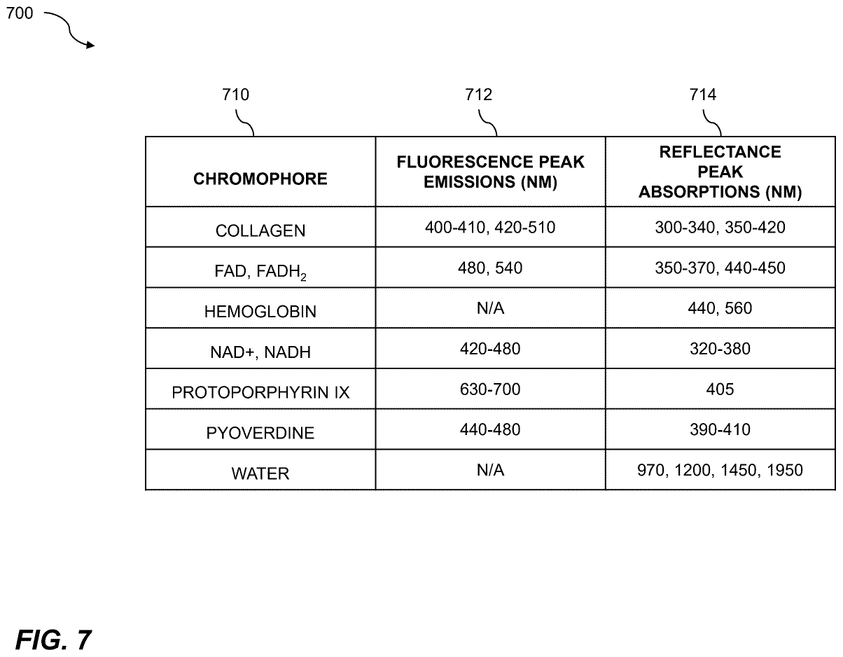

FIG. 7 is a table illustrating detected material sample compositions. Material sample compositions are provided by image analysis using topography-informed excitation and illumination. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

The table 700 includes various exemplar chromophores 710 that represent the detected material composition. The table 700 includes emission peaks for fluorescence excitation 712. The table 700 also includes absorption peaks for reflectance illumination 714. The fluorescence peak emissions of a chromophore desired to be detectable can be analyzed to determine variations between a first excitation wavelength and a second excitation wavelength. In embodiments, the fluorescence excitation wavelengths are 365 nm and 405 nm, which can optimize detection results when using only two excitation wavelengths to enable capture of fluorescence signatures across a broad variety of chromophores. In addition, reflectance characteristics can be integrated with fluorescence excitations to further refine a particular detection signature. Furthermore, the reflectance characteristics can be analyzed to determine variations among captured responses to three reflectance illuminations, which can enable chromophore detectability. In embodiments, the reflectance illumination wavelengths are 475 nm, 530 nm, and 665 nm, which can optimize detection results when using only three illumination wavelengths to enable capture of reflectance signatures across a broad variety of chromophores. It should be noted, however, that while exact wavelengths of commercially available LED light sources will vary, nonetheless, an LED light source is typically marketed and sold with a singular, named wavelength, which wavelength is used in descriptions herein. Any particular wavelength can be substantially indicated by the abovementioned wavelengths when the particular wavelength is within 5 nm to 10 nm of the abovementioned wavelengths. Furthermore, the light sources can be sequenced such that only one wavelength at a time impinges on the material sample, which can reduce unwanted light contamination. However, certain material compositions may be detectable even when light sources are used in combination.

Referring again to table 700, it can be seen that the chromophore collagen shows emission peaks of 400 nm to 410 nm for a 365 nm excitation and 420 nm to 510 nm for a 405 nm excitation. Collagen also shows absorption peaks at 300 nm to 340 nm and 350 nm to 420 nm. The chromophore FADH₂ shows emission peaks of 480 nm for a 365 nm excitation and 540 nm for a 405 nm excitation, while FAD shows absorption peaks at 350 nm to 370 nm and 440 nm to 450 nm (see FIG. 6 for additional details). The chromophore hemoglobin does not exhibit fluorescence at either fluorescence excitation wavelength, but exhibits absorption peaks at 440 nm and 560 nm. The chromophores NAD+ and NADH show emission peaks of 420 nm to 480 nm for a 365 nm excitation, although the NAD+ emission is much smaller, while NADH shows an absorption peak between 320 nm to 380 nm (see FIG. 5 for additional details). The chromophore protoporphyrin IX shows an emission peak of 630 nm to 700 nm for a 405 nm excitation, but no emission peaks for a 365 nm excitation, and an absorption peak of 405 nm. The chromophore pyoverdine shows an emission peak of 440 nm to 480 nm for a 405 nm excitation, but no emission peaks for a 365 nm excitation, and an absorption peak of 390 nm to 410 nm. The chromophore water does not exhibit any emission peaks when excited with 365 nm or 405 nm light, but exhibits absorption peaks at 970 nm, 1200 nm, 1450 nm, and 1950 nm.

Furthermore, the fluorescence excitation wavelength signatures can be additionally leveraged by factoring in a chromophore's peak absorptions. Two fluorescence excitation wavelengths, one with a spectral peak at 365 nm and another with a spectral peak at 405 nm, can be used to improve specificity in identifying particular autofluorescing chromophores. As an example, protoporphyrin IX (PPIX) has an absorption peak at 405 nm that decreases drastically in moving to the blue region of the spectrum. With a 405 nm excitation, a large fluorescence response signal is anticipated between 630 nm and 700 nm. However, with a 365 nm excitation, the response signal will decrease drastically because PPIX absorption is so much less at 365 nm. A similar, but inverse, effect is seen for Flavin Adenine Dinucleotide (FAD), which has absorption peaks at 350 nm to 370 nm and 440 nm to 450 nm. When excited at 365 nm, the fluorescence response signal will be substantially larger than a fluorescence response signal excited at 405 nm, because it falls in an absorption trough between the aforementioned FAD absorption peaks of 350 nm to 370 nm and 440 nm to 450 nm.

Continuing on in table 700, to elucidate reflectance signature analysis for chromophore material detection, diffuse reflection can be monitored at three different illumination wavelengths: 480 nm, 540 nm and 670 nm. Because a molecule's absorption spectrum does not predict its fluorescence intensity, diffuse reflectance can be used to confer chromophore detection specificity separate from fluorescence. By way of example, in the case of hemoglobin, a distinct trough at 480 nm can be probed relative to the peak at ˜550 nm. Thus, a higher reflectance signal at 480 nm than at ˜550 nm would be a strong indicator of the presence of hemoglobin. Furthermore, a marked decrease in absorption, going from the peak at ˜550 nm to the isosbestic point at 808 nm, would enable additional detection specificity. In the case of collagen or melanin, distinct increases in diffuse reflectance would be observed in going from 480 nm to 540 nm to 670 nm. As a final example, for FAD, which absorbs at 480 nm, but not at 540 nm or 670 nm, a weaker diffuse reflection signal would be expected at the bluest of the three wavelengths, relative to those at 540 nm and 670 nm, for which absorption is minimal and diffuse reflectance signal is, conversely, relatively high.

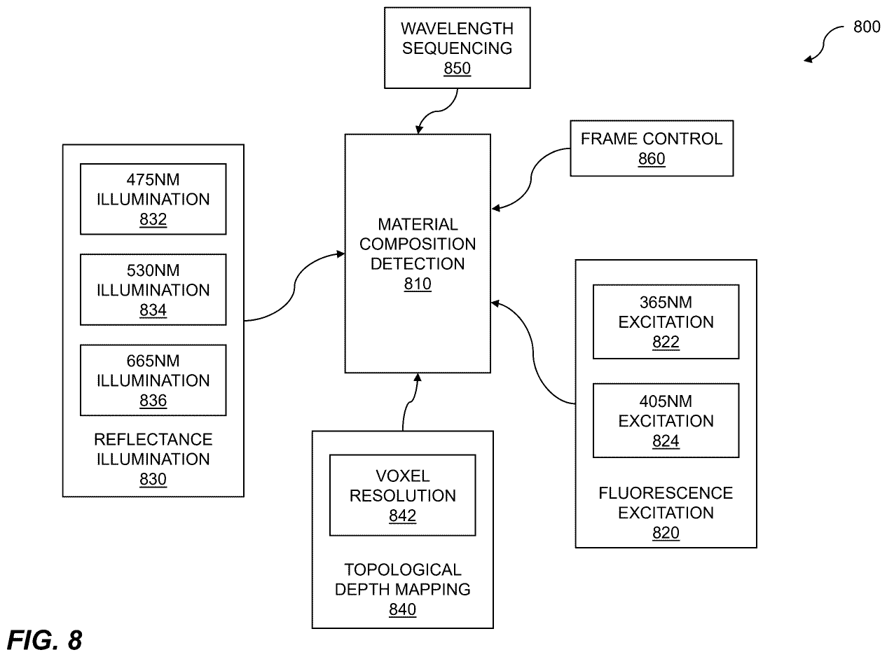

FIG. 8 is a system block diagram for depth-compensated image analysis using multiple light signatures. Image analysis using topography-informed excitation and illumination is disclosed. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

The system block diagram 800 includes a material composition detection component 810. The material composition detection component can analyze the responses to one or more light wavelength sources used to excite and/or illuminate a material sample. The material sample can include a skin wound, the exudate of the skin wound, and so on. The one or more light bandwidth sources can include far-infrared, mid-infrared, and near-infrared sources; visible light sources such as red, green, and blue sources; and so on. The analysis can include determining a material composition based on one or more responses of the material sample to the light sources. The responses can include fluorescence characteristics and reflectance characteristics, and they can be captured by an image sensor (described elsewhere).

The system block diagram 800 includes a fluorescence excitation component 820. The fluorescence excitation component can source at least two light wavelengths that impinge on a material sample and cause a fluorescence response. The exciting enables capture of fluorescence characteristics of the material sample. The fluorescence characteristics can include light wavelengths within one or more of ultraviolet light wavelengths, infrared light wavelengths, visible light wavelengths, and so on. The material sample can exhibit fluorescence characteristics along the Red-Green-Blue (RGB) light wavelength spectrum. The fluorescence characteristics can be captured using a sensor such as an RGB sensor. In embodiments, the light wavelengths that excite fluorescence characteristics of the material sample can be substantially a 365 nm excitation 822 light wavelength and a 405 nm excitation 824 light wavelength.

The system block diagram 800 includes a reflectance illumination component 830. The reflectance illumination component can source at least three additional light wavelengths that impinge on a material sample and cause a reflectance response commensurate with the absorption characteristics of the material sample. The additional light wavelengths can include infrared light wavelengths, visible light wavelengths, etc. In embodiments, the at least three additional light wavelengths can include a blue-band light wavelength, a green-band light wavelength, and a red-band light wavelength. The blue-, green-, and red-band light can comprise various light wavelengths. In embodiments, the blue-band light wavelength can be substantially a 475 nm illumination 832 light wavelength; the green-band light wavelength can be substantially a 530 nm illumination 834 light wavelength; and the red-band light wavelength can be substantially a 665 nm illumination 836 light wavelength. Other additional light wavelengths can be used. Further embodiments can include illuminating the material sample with at least one further additional light wavelength. The at least one further additional light wavelength can include infrared light, visible light, etc. In embodiments the at least one further additional light wavelength can include an infrared-band light wavelength. Various infrared-band light wavelengths can be used. In embodiments, the infrared-band light wavelength can be substantially a 940 nm wavelength. The illuminating enables capture of reflectance characteristics of the material sample. The reflectance characteristics can include infrared light, visible light, and the like. The material sample can exhibit reflectance characteristics along the Red-Green-Blue (RGB) light wavelength spectrum. The reflectance characteristics can be captured using a sensor such as an RGB sensor.

The system block diagram 800 includes a topological depth mapping component 840. The topological depth mapping component can generate an output indicative of the topography of the material sample. Having an accurate topography can enable characteristics corrections based on inverse square law light attenuation compensation. The topological depth mapping can employ voxel resolution 842 to communicate the depth mapping in a defined data structure, although other data structures are possible. The reflectance characteristics and the fluorescence characteristics intensities are modified based on the topological depth mapping to enable a more accurate material composition detection.

The system block diagram 800 includes a wavelength sequencing component 850. The wavelength sequencing component provides temporal sequencing of the at least two excitation light wavelengths and the at least three additional illumination light wavelengths. Sequencing the source light wavelengths can help eliminate cross-wavelength sample responses and enable more accurate material composition detection. In other words, the material sample responses to one particular excitation or illumination wavelength will not be skewed by impingement on the material sample of other excitation or illumination wavelengths. In embodiments, the sequencing mitigates cross-wavelength material sample responses.

In addition, the wavelength sequencing can be synchronized with the image capturing of the image sensor using a frame control component 860.

Synchronization of the image frames or video frames of an integrated smartphone camera can be achieved by using the smartphone's integrated flash LED to trigger and synchronize the sequenced excitation and illumination wavelengths to the smartphone camera frames. For example, an asynchronous user command, such as a software application “button” push, can initiate the smartphone camera flash and frame recording. This initial camera flash is detected and used to trigger the flashing of the sequenced excitation wavelength and illumination wavelength LEDs, which are timed to synchronize to the smartphone camera frames, based on the timing of the particular smartphone model, i.e., using the smartphone's built-in flash/frame synchronization parameters. In addition, because the first frame recorded by the smartphone is illuminated by the unpolarized smartphone LED, the first image comprises a standard RGB image that includes specular reflection, which is valuable for providing monocular depth estimation, but not helpful for capturing subsequent fluorescence and reflectance responses. The frame control component causes a wavelength excitation and/or illumination to occur when a camera frame in the RGB image sensor used to capture fluorescence characteristics and reflectance characteristic is actively sampling the light it receives. Thus, for a series of single images, a burst of single images, and/or the images of a video, the frame component ensures that the response of the material sample to the excitation and/or illumination wavelength(s) is captured, along with the initial, standard “white light” flash LED response used for monocular depth estimation. In embodiments, the sequencing is synchronized to camera frames of a device providing image capture of the fluorescence characteristics and the reflectance characteristics of the material sample. In embodiments, the synchronization is triggered by detection of a flash from the device providing the image capture.

The frame control can alternatively, or additionally, use three or more frames of a video that are centered around each of the sequenced light wavelengths to provide an overfilled number of video frames that bracket the response to the wavelength of light being sequenced and shined on the material sample. The best frame from among the overfilled video frames can be selected for use by the material composition detection unit. Which frame is best can be determined by simply selecting the center frame of the overfilled video frame sequence, e.g., the middle frame of a three-frame sequence, or by using a more sophisticated algorithm or heuristic determined by sample type category, ambient lighting conditions, human use factors, and so on.

In embodiments, the at least two different fluorescence excitation wavelengths comprise substantially a 365 nm wavelength and substantially a 405 nm wavelength. In embodiments, the at least three additional light wavelengths comprise substantially a 475 nm wavelength, substantially a 530 nm wavelength, and substantially a 665 nm wavelength. Some embodiments comprise sequencing the fluorescence exciting wavelengths and the at least three additional light wavelengths. In embodiments, the sequencing aligns the fluorescence exciting wavelengths and the at least three additional light wavelengths to camera frames providing the capture. Some embodiments comprise overfilling the camera frames such that at least three adjacent frames in a video sequence are all illuminated by the same wavelength. Some embodiments comprise choosing a center frame to optimize the analysis.

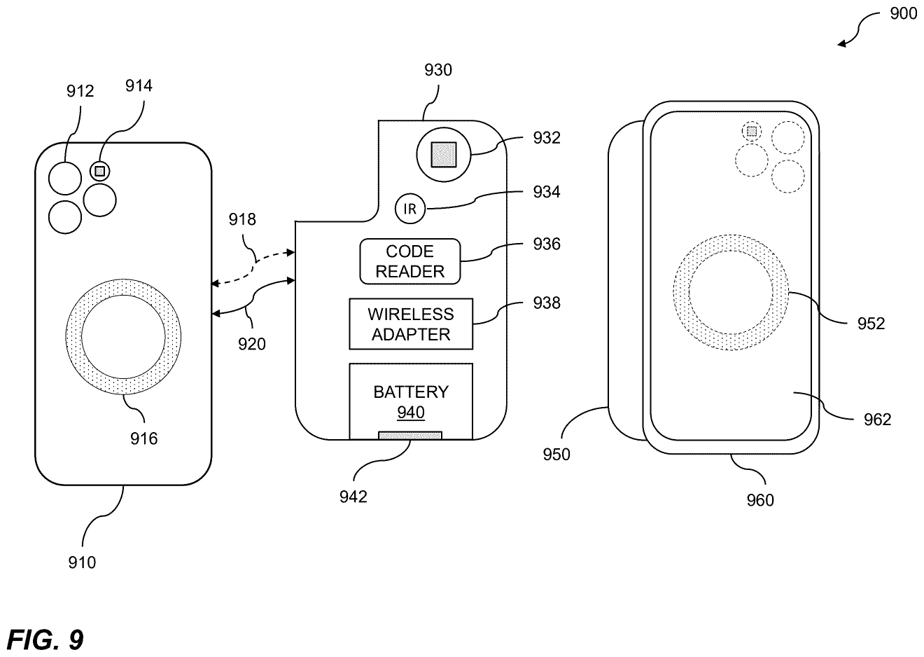

FIG. 9 is an infographic for depth-compensated image analysis using a smart device. Smart device image analysis can be based on topography-informed excitation and illumination. Fluorescence from a material sample is excited. The exciting enables capture of fluorescence characteristics of the material sample. The fluorescence characteristics occur along the Red-Green-Blue (RGB) light wavelength spectrum. The exciting is accomplished using at least two different fluorescence excitation wavelengths. The material sample is illuminated with additional light wavelengths. The illuminating enables capture of reflectance characteristics of the material sample. The material sample exhibits reflectance characteristics along the RGB light wavelength spectrum. The illuminating is accomplished using at least three additional light wavelengths. A depth estimation of the material sample is mapped, based on the reflectance characteristics. A material composition is detected, based on analysis of the fluorescence characteristics, the reflectance characteristics, and the depth estimation. The material composition is indicative of a biophysical status of the material sample.

The infographic 900 shows a smart device, in this case a smartphone, and a device for material sample image analysis that can be coupled to the smartphone. The device can be coupled to the smartphone using various techniques such as wired techniques, wireless techniques, hybrid wired and wireless techniques, and so on. The infographic 900 includes a back of a smartphone 910. The back of the smartphone can include one or more cameras or light sensing components 912. The cameras or light sensing components can include one or more of a normal lens, a macro (closeup) lens, a telephoto (distance) lens, and so on. While three lenses are shown, the smartphone can include one lens, two lenses, etc. The back of the smartphone can include a light source 914. The light source can include a visible light source, an infrared light source, or other light source. The light source can include a strobed source such as a photographic flash, a continuous light source such as a phone flashlight, and the like. The light source 914 can be used in the mapping of a depth estimation, as previously described. The back of the smartphone can further include coupling 916 such as a magnetic coupling. The coupling can be used to couple the back of the smartphone to a wound care image analysis device. In embodiments, the coupling can be used to transfer power from the device to the smartphone.

The infographic 900 shows a wound care image analysis device 930 that can be coupled to the smartphone 910. The device can include a sleeve that can hold the smartphone, a backpack that couples to the back of the smartphone using the coupling 916, a “sidekick” device, and so on. In addition to the mechanical, magnetic, physical, etc. coupling of the device to the smartphone, coupling can include a wireless connection 918. The wireless connection can include a Bluetooth®, a near-field communication (NFC®), Zigbee®, Wi-Fi® such as 802.11, and so on. The coupling of the device to the smartphone can include a wired connection 920. The wired connection can include a serial protocol such as RS-232, a parallel such as IEEE-488®, an Ethernet® protocol, etc. The device can include a light source 932. In embodiments, the external light source can emit at least one light wavelength capable of illuminating a material sample. The external light source can include a visible light source, a light detection and ranging (LIDAR) light source, and the like. The wavelength can be chosen to excite a fluorescence response from a material sample illuminated by the light source. In embodiments, the external light source can emit at least one light wavelength capable of illuminating a material sample. The device can include an IR light source 934. The IR light source can include various IR wavelengths, such an infrared wavelength, a near-infrared (NIR) wavelength, and so on. The device can include a code reader 936. The code reader can be used to read machine-readable code such as a barcode, a quick-response (QR), and the like. The device can include a wireless adapter 938. The wireless adapter can enable communication between the device and the smartphone, a network such as a computer network, a server, etc. The wireless adapter can include a Wi-Fi® adapter. The device 930 can include a battery 940. The battery can be used to power the device, to provide power to a smartphone coupled to the device, and so on. In embodiments, the external light source can be powered by a colocated battery. The device can include a power port 942. The power port can be used to charge the battery 940, power the device, etc. In embodiments, charging the colocated battery enables charging an integrated battery of the smartphone.

The infographic 900 can include the device 950 coupled to the smartphone 960. The device is shown in its reversed orientation, and the phone shown with the display 962 forward. That is, the device is shown coupled to the back of the phone. The back of the device can include a coupling 952 that is compatible with the coupling 916 on the back of the phone. The coupling can include a mechanical coupling, a magnetic coupling, an electrical coupling, and so on. The coupling can secure the device to the phone. The device coupling and the phone coupling can be disconnected by pulling, twisting, sliding, etc. The device coupling can provide power to the phone via the phone coupling. In embodiments, the device coupling and the phone coupling can transfer data such light wavelength data, illumination signature data, device control data, and the like. Note that the “cutout” of the device enables clearance for the light source and the one or more cameras associated with the smartphone.

In embodiments, the fluorescence characteristics and the reflectance characteristics are captured using a commercially available smart device. In embodiments, the smart device comprises a smartphone, smart glasses, a smart watch, an augmented reality headset, or a virtual reality headset. Some embodiments comprise coupling a multi-wavelength light source to the smart device. In embodiments, the multi-wavelength light source enables the exciting and the illuminating. Some embodiments comprise triggering the multi-wavelength light source using an integrated light emitting diode (LED) in the smart device.

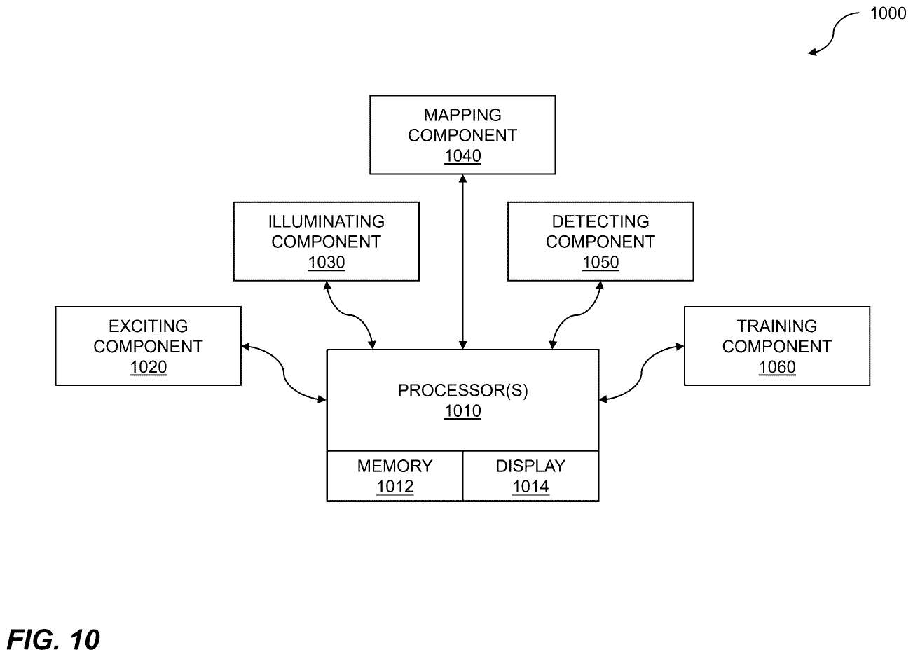

FIG. 10 is a system diagram for depth-compensated image analysis using multiple light signatures. The system 1000 can include one or more processors 1010, which are coupled to a memory 1012 which stores instructions. The system 1000 can further include a display 1014 coupled to the one or more processors 1010 for displaying data, indications of sample analysis, illumination signatures, directions, input requests, control options, excitation wavelengths, filter options, compensation options, data forwarding options, and so on. Embodiments of the system 1000 comprise a computer system for image analysis comprising: one or more processors 1010 that are coupled to the memory 1012 which stores instructions, wherein the one or more processors, when executing the instructions which are stored, are configured to: excite fluorescence from a material sample, wherein the exciting enables capture of fluorescence characteristics of the material sample, wherein the fluorescence characteristics occur along the Red-Green-Blue (RGB) light wavelength spectrum, and wherein the exciting is accomplished using at least two different fluorescence excitation wavelengths; illuminate the material sample with additional light wavelengths, wherein the illuminating enables capture of reflectance characteristics of the material sample, wherein the material sample exhibits reflectance characteristics along the RGB light wavelength spectrum, and wherein the illuminating is accomplished using at least three additional light wavelengths; map a depth estimation of the material sample, based on the reflectance characteristics; and detect a material composition, wherein the material composition is indicative of a biophysical status of the material sample, and wherein the detecting is based on analysis of the fluorescence characteristics, the reflectance characteristics, and the depth estimation.

The system 1000 includes an exciting component 1020. The exciting component can be used to excite fluorescence from a material sample by scanning a plurality of optical excitation ultraviolet light wavelength bands on the sample. The material sample can exhibit fluorescence characteristics that occur along the Red-Green-Blue (RGB) light wavelength spectrum. The exciting can be accomplished using at least two different fluorescence excitation wavelengths. The material sample can comprise one or more materials, tissues, wounds, wound exudate, biologics, drugs, foods, agricultural products, and so on. In embodiments, the material sample can include cells, tissues, and organs. The material sample can be collected from a variety of cells, tissues, and organs associated with a patient, particularly from wound tissue. The material sample may be part of a patient's skin, completely attached to the patient. The material sample may be biomaterial collected from a patient, such as a sample of wound exudate. In a usage example, the material sample can include healthy tissue, damaged tissue, and so on. The optical excitation light wavelength bands can be provided by various sources including an LED light source, a laser light source, and so on. The light sources can emit narrow spectra of light at primarily two wavelengths in the ultraviolet spectrum. The excitation wavelengths can be targeted toward material sample fluorescence. A fluorescence excitation light wavelength signal can have a wavelength which is less than a wavelength of the RGB light wavelength spectrum. The wavelength less than a wavelength of the RGB light wavelength spectrum can be substantially between 200 nm and 450 nm. The wavelength bands can include a first band of the optical excitation light wavelength bands comprising wavelengths substantially in the range of 325 nm to 375 nm and a second band of the optical excitation light wavelength bands comprising wavelengths substantially in the range of 375 nm to 425 nm. In embodiments, the two different fluorescence excitation wavelengths comprise substantially a 365 nm wavelength and substantially a 405 nm wavelength.

The system 1000 includes an illuminating component 1030. The illuminating component 1030 can illuminate the material sample with additional light wavelengths beyond the aforementioned fluorescence excitation wavelengths. The illuminating enables capture of reflectance characteristics of the material sample. The material sample exhibits reflectance characteristics along the Red-Green-Blue (RGB) light wavelength spectrum. The three additional light wavelengths can include far-infrared (FIR), mid-infrared (MIR), and near-infrared (NIR); visible light; and so on. In embodiments, the at least three additional light wavelengths can include a blue-band light wavelength, a green-band light wavelength, and a red-band light wavelength. The blue-band light, the green-band light, and the red-band light can include various wavelengths of blue, green, and red light, respectively. The illuminating component can be used to scan a plurality of optical excitation visible light wavelength bands on a material sample, where the material sample exhibits optical spectral characteristics along the light wavelength spectrum. The material sample can comprise one or more materials, tissues, wounds, wound exudate, biologics, drugs, foods, agricultural products, and so on. The optical excitation light wavelength bands can be provided by various sources including an incandescent light source, an LED light source, a laser light source, and so on. The light sources can emit narrow spectra of light at various peak wavelengths across the visible light (RGB) spectrum. The illumination wavelengths can be targeted toward material sample reflectance. A reflectance excitation light wavelength signal can have a wavelength contained within the wavelengths of the RGB light wavelength spectrum, i.e., approximately 400 nm to approximately 700 nm. Note that there are varying definitions of the exact wavelengths comprising the RGB, or visible light, spectrum. However, whatever those definitions are, they do not override the wavelengths described in the techniques herein. A first illumination wavelength can be substantially 475 nm. A second illumination wavelength can be substantially 530 nm. A third illumination wavelength can be substantially 665 nm. Additional illumination wavelengths can also be used.

The system 1000 can include a mapping component 1040. The mapping component can map a depth estimation across the x-y image of the material sample. The depth estimation describes the topography of the material sample and can be used to compensate for inverse square law losses of light energy that are captured. The capturing can be performed by an imaging device, such as the camera of a smartphone or other RGB image capture sensor. The light that is captured can include the reflectance characteristics of the material sample. The mapping component can be accomplished using a monocular optics system. The depth estimation can be accomplished using a machine learning model. The depth estimation can be performed by illuminating the material sample with a white-light LED, such as the flashlight or camera flash LED integrated into most smartphones. The mapping can be used to more accurately analyze the fluorescence characteristics and the reflectance characteristics of the material sample, based on the compensating.

The system 1000 can include a detecting component 1050. The detecting component can detect a material composition, based on an analysis of the captured fluorescence characteristics, the captured reflectance characteristics, and the compensating depth estimation. The analysis can be accomplished through hard-coded circuitry, algorithms, lookup tables, machine learning, and so on. The analysis can be indicative of a biophysical status of the material sample. The biophysical status can include wound healing metrics, biomolecule concentrations, drug composition, drug potency, crop health, and food nutritional information, to name just a few.

The system 1000 can include a training component 1060. The training component can be used to train a machine learning model to enable a monocular optics system to provide accurate depth estimation and sample topography. The training can be based on using depth-enabled material sample images captured by a depth-enabled (3D) camera with monocular images of the same material sample. Various machine learning models can be used, such as neural networks, support vector machines, distributed neural networks, and so on. The training component can also be used to train additional machine learning models to help analyze and/or interpret the material composition and/or the biophysical status.

The system 1000 can include a computer program product embodied in a non-transitory computer readable medium for image analysis, the computer program product comprising code which causes one or more processors to perform operations of: exciting fluorescence from a material sample, wherein the exciting enables capture of fluorescence characteristics of the material sample, wherein the fluorescence characteristics occur along the Red-Green-Blue (RGB) light wavelength spectrum, and wherein the exciting is accomplished using at least two different fluorescence excitation wavelengths; illuminating the material sample with additional light wavelengths, wherein the illuminating enables capture of reflectance characteristics of the material sample, wherein the material sample exhibits reflectance characteristics along the Red-Green-Blue (RGB) light wavelength spectrum, and wherein the illuminating is accomplished using at least three additional light wavelengths; mapping a depth estimation of the material sample, based on the reflectance characteristics; and detecting a material composition, wherein the material composition is indicative of a biophysical status of the material sample, and wherein the detecting is based on analysis of the fluorescence characteristics, the reflectance characteristics, and the depth estimation.

Each of the above methods may be executed on one or more processors on one or more computer systems. Embodiments may include various forms of distributed computing, client/server computing, and cloud-based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general-purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are limited to neither conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM); an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States, then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A method for image analysis comprising:
exciting fluorescence from a material sample, wherein the exciting enables capture of fluorescence characteristics of the material sample, wherein the fluorescence characteristics occur along the Red-Green-Blue (RGB) light wavelength spectrum, and wherein the exciting is accomplished using at least two different fluorescence excitation wavelengths;
illuminating the material sample with additional light wavelengths, wherein the illuminating enables capture of reflectance characteristics of the material sample, wherein the material sample exhibits reflectance characteristics along the RGB light wavelength spectrum, and wherein the illuminating is accomplished using at least three additional light wavelengths;
mapping a depth estimation of the material sample, based on the reflectance characteristics; and
detecting a material composition, wherein the material composition is indicative of a biophysical status of the material sample, and wherein the detecting is based on analysis of the fluorescence characteristics, the reflectance characteristics, and the depth estimation.

2. The method of claim 1 wherein the mapping a depth estimation is accomplished using a monocular optics system.

3. The method of claim 2 wherein illumination for the monocular optics system is provided by an integrated smartphone LED.

4. The method of claim 2 wherein the depth estimation enables inverse square law correction of the fluorescence characteristics and the reflectance characteristics.

5. The method of claim 4 wherein the inverse square law correction enables depth resolution of three-dimensional (3D) material sample features.

6. The method of claim 5 wherein the 3D material sample features include tissue topology.

7. The method of claim 6 wherein the tissue topology is captured by voxels.

8. The method of claim 1 wherein the depth estimation is accomplished using a machine learning model.

9. The method of claim 8 wherein the machine learning model is trained using monocular RGB images compared with depth-enabled images.

10. The method of claim 9 wherein the depth enabled images are captured with a depth-capable camera.

11. The method of claim 1 wherein the at least two different fluorescence excitation wavelengths comprise substantially a 365 nm wavelength and substantially a 405 nm wavelength.

12. The method of claim 11 wherein the at least three additional light wavelengths comprise substantially a 475 nm wavelength, substantially a 530 nm wavelength, substantially a 665 nm wavelength.

13. The method of claim 12 further comprising sequencing the at least two different fluorescence exciting wavelengths and the at least three additional light wavelengths.

14. The method of claim 13 wherein the sequencing is synchronized to camera frames of a device providing image capture of the fluorescence characteristics and the reflectance characteristics of the material sample.

15. The method of claim 14 wherein the synchronization is triggered by detection of a flash from the device providing the image capture.

16. The method of claim 1 wherein the fluorescence characteristics and the reflectance characteristics are captured using a commercially available smart device.

17. The method of claim 16 further comprising coupling a multi-wavelength light source to the smart device.

18. The method of claim 17 wherein the multi-wavelength light source enables the exciting and the illuminating.

19. The method of claim 18 further comprising triggering the multi-wavelength light source using an integrated light emitting diode (LED) in the smart device.

20. The method of claim 1 further comprising using a time-of-flight sensor to calibrate the depth estimation.

21. The method of claim 1 wherein the material sample comprises a tissue sample.

22. A computer program product embodied in a non-transitory computer readable medium for image analysis, the computer program product comprising code which causes one or more processors to perform operations of:
exciting fluorescence from a material sample, wherein the exciting enables capture of fluorescence characteristics of the material sample, wherein the fluorescence characteristics occur along the Red-Green-Blue (RGB) light wavelength spectrum, and wherein the exciting is accomplished using at least two different fluorescence excitation wavelengths;
illuminating the material sample with additional light wavelengths, wherein the illuminating enables capture of reflectance characteristics of the material sample, wherein the material sample exhibits reflectance characteristics along the RGB light wavelength spectrum, and wherein the illuminating is accomplished using at least three additional light wavelengths;
mapping a depth estimation of the material sample, based on the reflectance characteristics; and
detecting a material composition, wherein the material composition is indicative of a biophysical status of the material sample, and wherein the detecting is based on analysis of the fluorescence characteristics, the reflectance characteristics, and the depth estimation.

23. A computer system for image analysis comprising:
a memory which stores instructions;
one or more processors coupled to the memory, wherein the one or more processors, when executing the instructions which are stored, are configured to:
excite fluorescence from a material sample, wherein the exciting enables capture of fluorescence characteristics of the material sample, wherein the fluorescence characteristics occur along the Red-Green-Blue (RGB) light wavelength spectrum, and wherein the exciting is accomplished using at least two different fluorescence excitation wavelengths;
illuminate the material sample with additional light wavelengths, wherein the illuminating enables capture of reflectance characteristics of the material sample, wherein the material sample exhibits reflectance characteristics along the RGB light wavelength spectrum, and wherein the illuminating is accomplished using at least three additional light wavelengths;
map a depth estimation of the material sample, based on the reflectance characteristics; and
detect a material composition, wherein the material composition is indicative of a biophysical status of the material sample, and wherein the detecting is based on analysis of the fluorescence characteristics, the reflectance characteristics, and the depth estimation.
